(19) United States
(12) Patent Application Publication
(10) Pub. No.: US 2025/0312109 A1
(43) Pub. Date: Oct. 9, 2025

(54) MULTI-INSTRUMENT COOPERATIVE FLEXIBLE URETEROSCOPE SURGICAL ROBOT

(71) Applicant: FUZHOU UNIVERSITY, Fuzhou (CN)

(72) Inventors: Zhen DENG, Fuzhou (CN); Jinbao HUANG, Fuzhou (CN); Shuai HE, Fuzhou (CN); Zhidong ZHAO, Fuzhou (CN); Bingwei HE, Fuzhou (CN); Jianhui CHEN, Fuzhou (CN); Shaoxing ZHU, Fuzhou (CN)

(21) Appl. No.: 18/938,514

(22) Filed: Nov. 6, 2024

Related U.S. Application Data
(63) Continuation of application No. PCT/CN2024/097212, filed on Jun. 4, 2024.

(30) Foreign Application Priority Data
Apr. 8, 2024 (CN) 202410413145.8

(51) Int. Cl. A61B 34/30; A61B 1/307; A61M 25/01

(52) U.S. Cl. CPC A61B 34/30; A61B 1/307; A61B 2034/301; A61B 2034/303; A61M 25/0113

(57) ABSTRACT

A multi-instrument cooperative flexible ureteroscope surgical robot is provided, which relates to the field of medical surgical robots. The system specifically includes a flexible ureteroscope driving device, a ureteral guide sheath driving device, a flexible ureteroscope rapid assembly device, a sheath rapid assembly device, a guide wire feeding device, a control box and other components. Firstly, the ureteral guide sheath driving device accurately positions a ureteral guide sheath into the urethra and ureter of the patient. Then, the flexible ureteroscope driving device drives a disposable flexible ureteroscope to enter into the body through the positioned ureteral guide sheath, and the guide wire feeding device controls the introduction of the laser guide wire along the flexible ureteroscope. In addition, a lifter provides necessary height adjustment according to the patient's physical condition. The control box provides an intuitive operation interface for surgeons.

### CROSS-REFERENCE TO RELATED APPLICATIONS

This application is a continuation of PCT/CN2024/097212, filed Jun. 4, 2024 and claims priority of Chinese Patent Application No. 202410413145.8, filed on Apr. 8, 2024, the contents of which are hereby incorporated by reference.

### TECHNICAL FIELD

The present disclosure relates to the field of medical surgical robots, and in particular to a multi-instrument cooperative flexible ureteroscope surgical robot.

### BACKGROUND

Flexible ureteroscope lithotripsy is a minimally invasive medical method for the treatment of ureteral calculi. In the traditional flexible ureteroscope lithotripsy, the doctor inserts an elongate flexible ureteroscope through the patient's urethra until it reaches the position of calculi. With the help of the camera on the flexible ureteroscope, the doctor may directly observe the calculi, and then use laser or mechanical tools to break the calculi into small pieces, and then remove or naturally excrete the fragments of calculi. Compared with open operation, this method has the advantages of less trauma and faster recovery. However, the existing flexible ureteroscope lithotripsy equipment has a certain limitation. Firstly, the success of the operation highly relies on the doctor's technical level and experience, and the accuracy and safety in the operation process require the doctor's operation level. Secondly, the manual operation of the flexible ureteroscope may lead to long operation time, discomfort for patients and increase the risk of complications. In addition, the doctor's vision is limited during the operation, so it may sometimes be difficult to accurately locate the calculi.

In view of this, how to provide an auxiliary device that may completely or partially solve the above technical problems is an urgent technical problem for those in the art.

### SUMMARY

An objective of the present disclosure is to provide a multi-instrument cooperative flexible ureteroscope surgical robot to solve the problems existing in the prior art.

In order to achieve the above objectives, the present disclosure provides a following solution: a multi-instrument cooperative flexible ureteroscope surgical robot, which includes:
a bottom plate, a first side plate and a second side plate are vertically arranged at two ends of the bottom plate respectively;
a flexible ureteroscope support plate, a first hinge plate and a second hinge plate are vertically arranged at two ends of the flexible ureteroscope support plate respectively, the flexible ureteroscope support plate is located between the first side plate and the second side plate, and the first hinge plate and the second hinge plate are respectively hinged with the first side plate and the second side plate;
a flexible ureteroscope rotating mechanism, the flexible ureteroscope rotating mechanism is fixedly connected with the bottom plate, an output end of the flexible ureteroscope rotating mechanism penetrates through the first side plate and is fixedly connected with the first hinge plate, and the flexible ureteroscope rotating mechanism is capable of driving the flexible ureteroscope support plate to rotate; and
a flexible ureteroscope feeding guide rail, the flexible ureteroscope feeding guide rail is fixedly arranged on the flexible ureteroscope support plate, and a flexible ureteroscope bending mechanism is arranged on the flexible ureteroscope feeding guide rail, and the flexible ureteroscope bending mechanism is slidably connected with the flexible ureteroscope feeding guide rail; a disposable flexible ureteroscope is arranged on the flexible ureteroscope bending mechanism, and the flexible ureteroscope bending mechanism is capable of adjusting a bending degree of the disposable flexible ureteroscope; and a flexible ureteroscope tube of the disposable flexible ureteroscope penetrates through the second hinge plate and the second side plate and is in communication with a ureteral guide sheath.

In an embodiment, the flexible ureteroscope rotating mechanism includes:
a flexible ureteroscope rotating motor, the flexible ureteroscope rotating motor is fixedly arranged on a lower surface of the bottom plate;
a driving pulley, an output end of the flexible ureteroscope rotating motor is in transmission connection with the driving pulley; and
a driven pulley, the driving pulley is in transmission connection with the driven pulley through a synchronous belt, and the driven pulley is fixedly connected with the first hinge plate, and a middle part of each of the first side plate, the second side plate, the first hinge plate and the second hinge plate is provided with a through hole, and the flexible ureteroscope tube of the disposable flexible ureteroscope extends from the through hole and is in communication with the ureteral guide sheath.

In an embodiment, the robot further includes:
a flexible ureteroscope rapid assembly device, the disposable flexible ureteroscope and the flexible ureteroscope bending mechanism are both arranged on the flexible ureteroscope rapid assembly device, and the flexible ureteroscope rapid assembly device is slidably connected with the flexible ureteroscope feeding guide rail.

In an embodiment, the robot further includes:
a flexible ureteroscope feeding motor, the flexible ureteroscope feeding motor is fixedly arranged on a lower surface of the flexible ureteroscope support plate; and the flexible ureteroscope feeding motor drives the flexible ureteroscope rapid assembly device to slide along the flexible ureteroscope feeding guide rail through a flexible ureteroscope feeding screw.

In an embodiment, the flexible ureteroscope bending mechanism includes:
a flexible ureteroscope bending motor, the flexible ureteroscope bending motor is fixedly arranged on the flexible ureteroscope rapid assembly device, the disposable flexible ureteroscope is provided with a bending knob capable of adjusting the bending degree, and an output end of the flexible ureteroscope bending motor is in transmission connection with the bending knob.

In an embodiment, the flexible ureteroscope rapid assembly device includes:
a base, a left clamping plate and a right clamping plate are arranged on the base, and a tension spring is arranged in the base, and two ends of the tension spring are fixedly connected with the left clamping plate and the right clamping plate respectively; and
a tension handle, the tension handle is in transmission connection with the left clamping plate and the right clamping plate; when the tension handle is pulled inward, the left clamping plate and the right clamping plate move towards each other and compress the tension spring, and the disposable flexible ureteroscope is capable of being fixed between the left clamping plate and the right clamping plate; when the tension handle is pulled outward, the tension spring resets and separates the left clamping plate and the right clamping plate.

In an embodiment, the robot further includes:
a mounting frame, the mounting frame is located below the bottom plate and is fixedly connected with the bottom plate;
a sheath tube feeding guide rail, the sheath tube feeding guide rail is arranged on the mounting frame;
a sheath tube fixing frame, the sheath tube fixing frame is arranged on the sheath tube feeding guide rail and is slidably connected with the sheath tube feeding guide rail, and the ureteral guide sheath is detachably connected with the sheath tube fixing frame, and a joint between the sheath tube fixing frame and the ureteral guide sheath is close to the second side plate; and
a sheath tube feeding motor, the sheath tube feeding motor is located below the mounting frame and is fixedly connected with the mounting frame, and the sheath tube feeding motor drives the sheath tube fixing frame to slide along the sheath tube feeding guide rail through a sheath tube feeding screw.

In an embodiment, the robot further includes a sheath tube rapid assembly device. The sheath tube rapid assembly device includes:
a lower cover, the lower cover is fixedly arranged on the sheath tube fixing frame;
an upper cover, the upper cover is hinged with the lower cover, and a clamping groove adapted to the ureteral guide sheath is defined between the upper cover and the lower cover; and
a cover lock, the cover lock is hinged with the lower cover, and when the upper cover rotates to an upper surface of the lower cover, the cover lock is capable of rotating to an upper surface of the upper cover.

In an embodiment, the robot further includes:
a support plate, the bottom plate is fixedly connected to the support plate, and a bottom of the support plate extends downward to form a support; and
a lifter, the lifter is in transmission connection with the support and is capable of driving the support to move in an up-and-down direction.

In an embodiment, the support plate has a clamping part, the bottom plate is fixed in the clamping part, and a clamping degree of the clamping part is adjusted by a tension knob.

In an embodiment, the robot further includes:
a worm gear motor, the worm gear motor is fixed on the disposable flexible ureteroscope through a guide wire motor fixing plate;
a lower roller, the lower roller is rotatably connected to the guide wire motor fixing plate through a lower roller cover, and the lower roller is fixedly connected with an output end of the worm gear motor;
an upper roller, the upper roller is rotatably connected to the guide wire motor fixing plate through an upper roller cover, the lower roller is rotatably connected to the upper roller, a groove is defined between the lower roller and the upper roller, and a laser guide wire penetrates through the groove, and the laser guide wire is driven to feed via rotation of the lower roller and the upper roller; and
a tee fitting, the tee fitting is in communication with the flexible ureteroscope tube of the disposable flexible ureteroscope, and the laser guide wire enters the flexible ureteroscope tube of the disposable flexible ureteroscope through the tee fitting. The laser guide wire is located between the two rollers, and is effectively pushed forward by the friction generated by the rollers to realize accurate feeding operation. When the guide wire needs to be recycled, the worm gear motor rotates reversely, and the guide wire is retracted backward, which greatly improves the flexibility and safety of the operation.

The present disclosure achieves the following technical effects.

Through the cooperation of multiple instruments, the flexible ureteroscope tube may be rotated, bent and translated, which greatly improves the flexibility and positioning accuracy of the equipment, reduces the operation time and reduces the risk of complications of patients.

As a communication tool between the patient's urethra and the flexible ureteroscope, the ureteral guide sheath may reduce the risk of direct contact between the flexible ureteroscope and the urethra and ureter, thus reducing the surgical trauma and reducing the patient's discomfort. At the same time, the ureteral guide sheath may also be used as an auxiliary positioning structure for the flexible ureteroscope, and the flexible ureteroscope may smoothly enter the patient's urethra along the ureteral guide sheath for surgical operation. In addition, when the flexible ureteroscope needs to be inserted twice in the operation, the ureteral guide sheath may be used continuously without secondary operation.

Both the flexible ureteroscope and the ureteral guide sheath are fixed by the rapid assembly device, so the fixing structure is stable and reliable, and the installation is convenient and rapid, which is conducive to simplifying preoperative preparation and improving the efficiency.

The lifter, the sheath tube feeding motor and the sheath tube feeding guide rail may flexibly adjust the height and position of the device to adapt to the physical conditions of various patients.

When the device disclosed in present disclosure is used for surgery, there is no need to make any wound or incision on the patient's body, and the flexible ureteroscope may be smoothly entered by using the human urethra, thus greatly reducing the intraoperative and postoperative pain for the patient.

### BRIEF DESCRIPTION OF THE DRAWINGS

In order to explain the embodiments of the present disclosure or the technical solution in the prior art more clearly, the drawings needed in the embodiments will be briefly introduced below. Apparently, the drawings in the following description are only some embodiments of the present disclosure. For one of ordinary skill in the art, other drawings may be obtained according to these drawings without paying creative effort.

FIG. 1 is a schematic diagram of an overall structure according to the present disclosure.

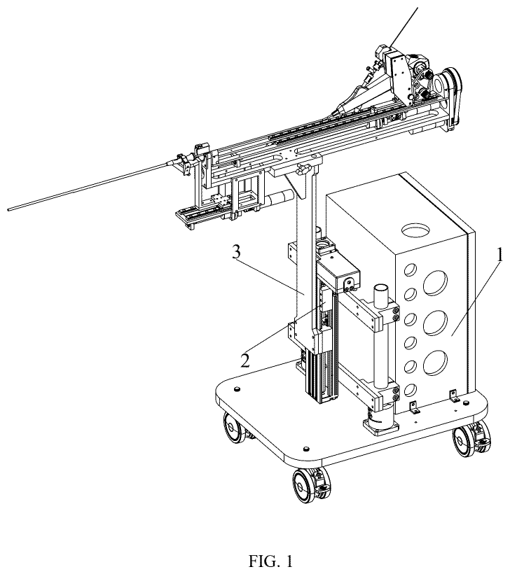

FIG. 2 is a schematic diagram of a flexible ureteroscope driving device according to the present disclosure.

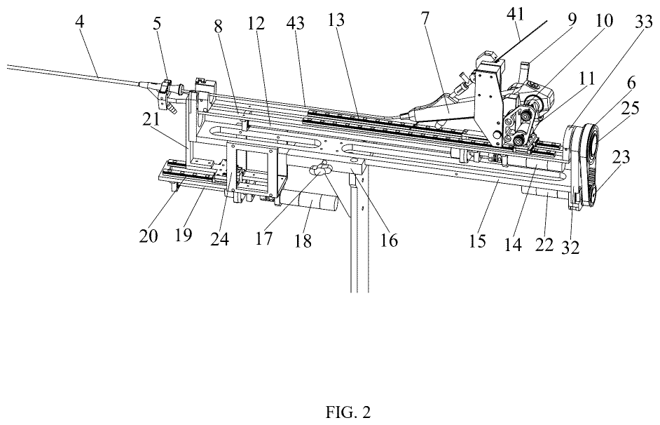

FIG. 3 is a schematic diagram of a laser guide wire driving device according to the present disclosure.

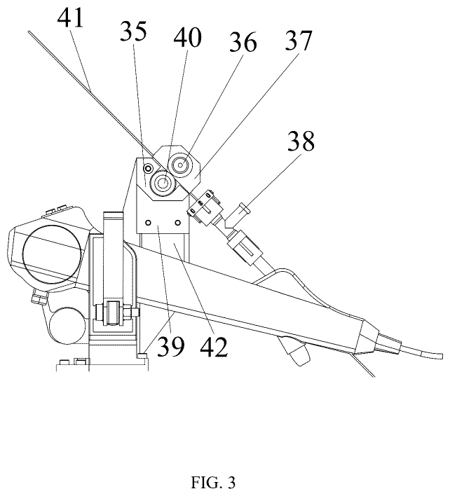

FIG. 4 is a schematic diagram of a flexible ureteroscope rapid assembly device according to the present disclosure.

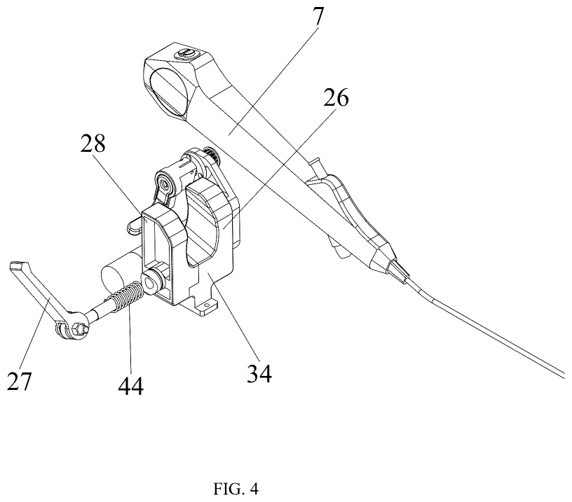

FIG. 5 is a schematic diagram of a sheath tube rapid assembly device according to the present disclosure.

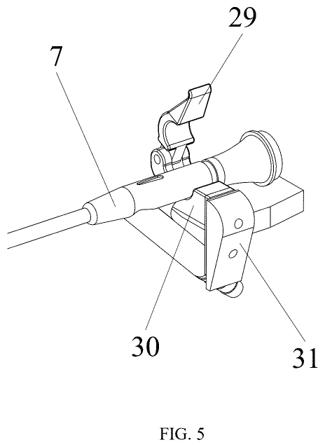

FIG. 6 is a schematic diagram of a sheath tube feeding guide rail according to the present disclosure.

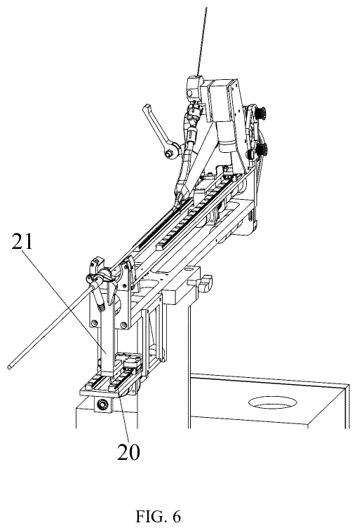

### DETAILED DESCRIPTION OF THE EMBODIMENTS

In the following, the technical solutions in the embodiments of the present disclosure will be clearly and completely described with reference to the attached drawings. Apparently, the described embodiments are only a part of the embodiments of the present disclosure, but not all the embodiments. Based on the embodiments in the present disclosure, all other embodiments obtained by one of ordinary skill in the art without creative effort belong to the protection scope of the present disclosure.

In order to make the above objects, features and advantages of the present disclosure more obvious and easier to understand, the present disclosure will be further described in detail with the attached drawings and specific embodiments.

In one embodiment, a multi-instrument cooperative flexible ureteroscope surgical robot is provided, which includes a bottom plate 15, a flexible ureteroscope support plate 8, a flexible ureteroscope rotating mechanism, and a flexible ureteroscope feeding guide rail 13. Two side plates 32 are vertically arranged at two ends of the bottom plate 15 respectively, and the bottom plate 15 and the two side plates 32 are partially hollowed out to facilitate wiring of following electric structures. Two hinge plates 33 are vertically arranged at two ends of the flexible ureteroscope support plate 8 respectively, the flexible ureteroscope support plate 8 is located between the two side plates 32. The side plates 32 is provided with a hinge hole near the top, and each of the two hinge plates 33 is hinged with corresponding one of the two side plates 32 through a hinge shaft. The flexible ureteroscope rotating mechanism is fixedly connected to the bottom plate 15, an output end of the flexible ureteroscope rotating mechanism penetrates through the side plate 32 and is fixedly connected with the hinge plate 33, and the flexible ureteroscope rotating mechanism is capable of driving the flexible ureteroscope support plate 8 to rotate. The flexible ureteroscope feeding guide rail 13 is fixedly arranged on the flexible ureteroscope support plate 8, and the flexible ureteroscope feeding guide rail 13 is provided with a flexible ureteroscope bending mechanism, and the flexible ureteroscope bending mechanism is slidably connected with the flexible ureteroscope feeding guide rail 13. A disposable flexible ureteroscope 7 is arranged on the flexible ureteroscope bending mechanism, and the flexible ureteroscope bending mechanism is capable of adjusting the bending degree of the disposable flexible ureteroscope 7, and a flexible ureteroscope tube 43 of the disposable flexible ureteroscope 7 penetrates through the hinge plate 33 and the side plate 32 and is in communication with a ureteral guide sheath 4.

In this embodiment, the flexible ureteroscope rotating mechanism includes a flexible ureteroscope rotating motor 22, a driving pulley 23 and a driven pulley 25. The flexible ureteroscope rotating motor 22 is fixedly arranged on the lower surface of the bottom plate 15. The output end of the flexible ureteroscope rotating motor 22 is in transmission connection with the driving pulley 23. The driving pulley 23 is in transmission connection with the driven pulley 25 through a synchronous belt 6, and the driven pulley 25 is fixedly connected with the hinge plate 33, and the middle part of each of the side plates 32 and the hinge plates 33 is provided with a through hole, and the flexible ureteroscope tube 43 of the disposable flexible ureteroscope 7 extends from the through hole and is in communication with the ureteral guide sheath 4.

In this embodiment, the robot also includes a flexible ureteroscope rapid assembly device 9. Both the disposable flexible ureteroscope 7 and the flexible ureteroscope bending mechanism are arranged on the flexible ureteroscope rapid assembly device 9, and the flexible ureteroscope rapid assembly device 9 is slidably connected with the flexible ureteroscope feeding guide rail 13.

In this embodiment, the robot also includes a flexible ureteroscope feeding motor 14. The flexible ureteroscope feeding motor 14 is fixedly arranged on the lower surface of the flexible ureteroscope support plate 8; and the flexible ureteroscope feeding motor 14 drives the flexible ureteroscope rapid assembly device 9 to slide along the flexible ureteroscope feeding guide rail 13 through a flexible ureteroscope feeding screw 12.

In this embodiment, the flexible ureteroscope bending mechanism includes a flexible ureteroscope bending motor 11. The flexible ureteroscope bending motor 11 is fixedly arranged on the flexible ureteroscope rapid assembly device 9, the disposable flexible ureteroscope 7 is provided with a bending knob 10 capable of adjusting the bending degree, and the output end of the flexible ureteroscope bending motor 11 is in transmission connection with the bending knob 10.

In this embodiment, the flexible ureteroscope rapid assembly device 9 includes a base 34 and a tension handle 27. A left clamping plate 28 and a right clamping plate 26 are arranged on the base 34, and a tension spring 44 is arranged in the base 34, and two ends of the tension spring 44 are fixedly connected with the left clamping plate 28 and the right clamping plate 26 respectively. The tension handle 27 is in transmission connection with the left clamping plate 28 and the right clamping plate 26; when the tension handle 27 is pulled inward, the left clamping plate 28 and the right clamping plate 26 move towards each other and compress the tension spring; when the tension handle 27 is pulled outward, the tension spring resets and separates the left clamping plate 28 and the right clamping plate 26. When the left clamping plate 28 and the right clamping plate 26 move towards each other, the disposable flexible ureteroscope 7 is capable of being fixed between the left clamping plate 28 and the right clamping plate 26. The tension handle is a handle used for moving the clamping plates towards or away from each other, the tension action of the handle on the clamping plates is achieved by the compress or loosen of the tension spring.

In this embodiment, the robot also includes a mounting frame 24, a sheath tube feeding guide rail 20, a sheath tube fixing frame 21, and a sheath tube feeding motor 18. The mounting frame 24 is located below the bottom plate 15 and is fixedly connected with the bottom plate 15. The sheath tube feeding guide rail 20 is arranged on the mounting frame 24. The sheath tube fixing frame 21 is arranged on the sheath tube feeding guide rail 20 and is slidably connected with the sheath tube feeding guide rail 20, and the ureteral guide sheath 4 is detachably connected with the sheath tube fixing frame 21, and the joint between the sheath tube fixing frame 21 and the ureteral guide sheath 4 is close to the side plate 32. The sheath tube feeding motor 18 is located below the mounting frame 24 and is fixedly connected with the mounting frame 24, and the sheath tube feeding motor 18 drives the sheath tube fixing frame 21 to slide along the sheath tube feeding guide rail 20 through a sheath tube feeding screw 19.

In this embodiment, the robot also includes a sheath tube rapid assembly device 5. The sheath tube rapid assembly device 5 includes: a lower cover 30, an upper cover 29 and a cover lock 31. The lower cover 30 is fixedly arranged on the sheath tube fixing frame 21. The upper cover 29 is hinged with the lower cover 30, and a clamping groove adapted to the ureteral guide sheath 4 is defined between the upper cover 29 and the lower cover 30. The cover lock 31 is hinged with the lower cover 30, and when the upper cover 29 rotates to the upper surface of the lower cover 30, the cover lock 31 is capable of rotating to the upper surface of the upper cover 29.

In this embodiment, the robot also includes a support plate 16 and a lifter 2. The bottom plate 15 is fixedly connected to the support plate 16, and the bottom of the support plate 16 extends downward to form the support 3. The lifter 2 is in transmission connection with the support 3 and is capable of driving the support 3 to move in an up-and-down direction. Further, the support plate 16 has a clamping part, the bottom plate 15 is fixed in the clamping part. The clamping degree of the clamping part is adjusted by a tension knob 17, and the bottom plate can be clamped or released by rotating the tension knob.

In this embodiment, the robot also includes a worm gear motor 42, a lower roller 40, an upper roller 36 and a tee fitting 38. The worm gear motor 42 is fixed on the disposable flexible ureteroscope 7 through a guide wire motor fixing plate 39.

The lower roller 40 is rotatably connected to the guide wire motor fixing plate 39 through a lower roller cover 35, and the lower roller 40 is fixedly connected to the output end of the worm gear motor 42.

The upper roller 36 is rotatably connected to the guide wire motor fixing plate 39 through an upper roller cover 37, the lower roller 40 is rotatably connected to the upper roller 36, a groove is defined between the lower roller 40 and the upper roller 36, and a laser guide wire penetrates through the groove, and the laser guide wire is driven to feed via the rotation of the lower roller 40 and the upper roller 36.

The first end of the tee fitting 38 is in communication with the flexible ureteroscope tube of the disposable flexible ureteroscope 7, the second end is connected to the laser guide wire, and the third end is connected to the water tube for supplying water to the kidneys. The laser guide wire enters the flexible ureteroscope tube of the disposable flexible ureteroscope 7 through the tee fitting 38.

In this embodiment, the robot also includes a control box 1 and a movable vehicle. The control box 1 is arranged on the movable vehicle and is responsible for controlling the operation of all motors. The lifting assembly (such as the lifter) is fixedly arranged on the movable vehicle, and the whole surgical robot may move synchronously with the movable vehicle.

The specific working process is as follows.
1. Installation of the ureteral guide sheath 4

According to the patient's physical condition, the height of the robot is adjusted by the

lifter 2. The tail end of the ureteral guide sheath 4 is fixed between the upper cover 29 and the lower cover 30 and pressed and locked by the cover lock 31. The sheath tube feeding motor 18 is started, and the sheath tube feeding motor 18 drives the sheath tube fixing frame 21 to slide along the sheath tube feeding guide rail 20 through the sheath tube feeding screw 19 to adjust the position of the ureteral guide sheath 4. The head end of the ureteral guide sheath 4 is positioned in the urethra and ureter of the patient to facilitate the smooth entry of the flexible ureteroscope.
2. Installation of the flexible ureteroscope

The flexible ureteroscope is fixed between the left clamping plate 28 and the right clamping plate 26. The flexible ureteroscope tube is introduced into the ureteral guide sheath 4.
3. Position adjustment of the flexible ureteroscope
3.1 Position adjustment of the flexible ureteroscope feed

The flexible ureteroscope feeding motor 14 is started, and the flexible ureteroscope feeding motor 14 drives the flexible ureteroscope rapid assembly device 9 to slide along the flexible ureteroscope feeding guide rail 13 through the flexible ureteroscope feeding screw 12 to adjust the feed position of the flexible ureteroscope.
3.2 Bending degree adjustment of flexible ureteroscope

The flexible ureteroscope bending motor 11 is started, and the flexible ureteroscope bending motor 11 drives the bending knob 10 to rotate to adjust the bending degree of the flexible ureteroscope.
3.3 Angle adjustment of flexible ureteroscope

The flexible ureteroscope rotating motor 22 is started, and the flexible ureteroscope rotating motor 22 sequentially drives the driving pulley 23, the driven pulley 25 and the hinge plate 33 to rotate, thereby driving the flexible ureteroscope support plate 8 and the flexible ureteroscope to rotate, so as to adjust the angle of the flexible ureteroscope.

After the position of the flexible ureteroscope is determined, the flexible ureteroscope lithotripsy may be performed normally.

In the above-mentioned process, the driving device corresponding to the ureteral guide sheath 4 and the driving device corresponding to the flexible ureteroscope cooperate dynamically. This cooperation not only includes the initial guidance of the flexible ureteroscope into the body by the ureteral guide sheath 4, but also the corresponding adjustment of the ureteral guide sheath 4 with the movement of the flexible ureteroscope during the operation to optimize the surgical path and improve the surgical safety.

At the beginning of the operation, the driving device corresponding to the ureteral guide sheath 4 is used for accurately positioning and inserting the ureteral guide sheath 4 into the urethra and ureter of the patient, ensuring an initial safe passage for the insertion and operation of the flexible ureteroscope. Once the ureteral guide sheath 4 reaches its initial position, the flexible ureteroscope is loaded and connected to the system through the flexible ureteroscope rapid assembly device, and then enters the body of the patient along the ureteral guide sheath 4.

When the flexible ureteroscope moves in the body and performs surgical operations such as lithotripsy, according to the real-time position of the flexible ureteroscope and the surgical requirements, the driving device corresponding to the ureteral guide sheath 4 adjusts the position and posture of the ureteral guide sheath 4. This dynamic adjustment allows the flexible ureteroscope to be more flexible in the ureter, while maintaining the safety and clear visibility of the surgical area, ensuring the efficient and accurate implementation of lithotripsy and other surgical operations.

This dynamic cooperation between the flexible ureteroscope and the ureteral guide sheath 4 significantly improves the flexibility and safety of the operation and reduces the risk of possible trauma and complications during the operation. By precisely controlling the position adjustment of the ureteral guide sheath 4 to respond to the movement of the flexible ureteroscope, the flexible ureteroscope operation is more efficient and safer, and a better treatment solution is provided for patients.
4. Laser guide wire feeding

The upper roller 36 and the lower roller 40 are respectively installed on corresponding cover plates, namely, the upper roller cover 37 and the lower roller cover 35. The groove is designed between the upper roller 36 and the lower roller for guiding the laser guide wire 41 to pass smoothly, and the length direction of the groove is parallel to the moving direction of the laser guide wire.

Before the operation, the laser guide wire 41 is placed in the groove formed by the upper roller 36 and the lower roller 40 to ensure that the laser guide wire 41 is correctly positioned and ready for feeding, and the worm gear motor 42 is started to drive the lower roller 40 to rotate. Due to the friction, the upper roller 36 will also follow the rotation. At this time, the laser guide wire 41 sandwiched between the two rollers begins to feed forward. During feeding, the laser guide wire 41 enters into the disposable flexible ureteroscope 7 through the channel of the tee fitting 38, realizing the stable feeding of the laser guide wire 41.

In the description of the present disclosure, it should be understood that the terms “longitudinal”, “transverse”, “up”, “down”, “front”, “back”, “left”, “right”, “vertical”, “horizontal”, “top”, “bottom”, “inside”, “outside”, etc. indicate orientation or positional relationships based on the orientation or positional relationships shown in the accompanying drawings, only for the convenience of describing the present disclosure, and do not indicate or imply that the device or element referred to must have a specific orientation, be constructed and operated in a specific orientation, and therefore cannot be understood as a limitation of the present disclosure.

The above-mentioned embodiments only describe the preferred mode of the present disclosure, and do not limit the scope of the present disclosure. Under the premise of not departing from the design spirit of the present disclosure, various modifications and improvements made by one of ordinary skill in the art to the technical solution of the present disclosure should fall within the protection scope of the present disclosure.

What is claimed is:

1. A multi-instrument cooperative flexible ureteroscope surgical robot, comprising:
a bottom plate, wherein a first side plate and a second side plate are vertically arranged at two ends of the bottom plate respectively;
a flexible ureteroscope support plate, wherein a first hinge plate and a second hinge plate are vertically arranged at two ends of the flexible ureteroscope support plate respectively, the flexible ureteroscope support plate is located between the first side plate and the second side plate, and the first hinge plate and the second hinge plate are respectively hinged with the first side plate and the second side plate;
a flexible ureteroscope rotating mechanism, wherein the flexible ureteroscope rotating mechanism is fixedly connected with the bottom plate, an output end of the flexible ureteroscope rotating mechanism penetrates through the first side plate and is fixedly connected with the first hinge plate, and the flexible ureteroscope rotating mechanism is capable of driving the flexible ureteroscope support plate to rotate; and
a flexible ureteroscope feeding guide rail, wherein the flexible ureteroscope feeding guide rail is fixedly arranged on the flexible ureteroscope support plate, and a flexible ureteroscope bending mechanism is arranged on the flexible ureteroscope feeding guide rail, and the flexible ureteroscope bending mechanism is slidably connected with the flexible ureteroscope feeding guide rail; a disposable flexible ureteroscope is arranged on the flexible ureteroscope bending mechanism, and the flexible ureteroscope bending mechanism is capable of adjusting a bending degree of the disposable flexible ureteroscope; and a flexible ureteroscope tube of the disposable flexible ureteroscope penetrates through the second hinge plate and the second side plate and is in communication with a ureteral guide sheath.

2. The robot of claim 1, wherein the flexible ureteroscope rotating mechanism comprises:
a flexible ureteroscope rotating motor, wherein the flexible ureteroscope rotating motor is fixedly arranged on a lower surface of the bottom plate;
a driving pulley, wherein an output end of the flexible ureteroscope rotating motor is in transmission connection with the driving pulley; and
a driven pulley, wherein the driving pulley is in transmission connection with the driven pulley through a synchronous belt, the driven pulley is fixedly connected with the first hinge plate, a middle part of each of the first side plate, the second side plate, the first hinge plate and the second hinge plate is provided with a through hole, and the flexible ureteroscope tube of the disposable flexible ureteroscope extends from the through hole and is in communication with the ureteral guide sheath.

3. The robot of claim 1, further comprising:
a flexible ureteroscope rapid assembly device, wherein the disposable flexible ureteroscope and the flexible ureteroscope bending mechanism are both arranged on the flexible ureteroscope rapid assembly device, and the flexible ureteroscope rapid assembly device is slidably connected with the flexible ureteroscope feeding guide rail.

4. The robot of claim 3, further comprising:
a flexible ureteroscope feeding motor, wherein the flexible ureteroscope feeding motor is fixedly arranged on a lower surface of the flexible ureteroscope support plate; and the flexible ureteroscope feeding motor drives the flexible ureteroscope rapid assembly device to slide along the flexible ureteroscope feeding guide rail through a flexible ureteroscope feeding screw.

5. The robot of claim 3, wherein the flexible ureteroscope bending mechanism comprises:
a flexible ureteroscope bending motor, wherein the flexible ureteroscope bending motor is fixedly arranged on the flexible ureteroscope rapid assembly device, the disposable flexible ureteroscope is provided with a bending knob capable of adjusting the bending degree, and an output end of the flexible ureteroscope bending motor is in transmission connection with the bending knob.

6. The robot of claim 3, wherein the flexible ureteroscope rapid assembly device comprises:
a base, wherein a left clamping plate and a right clamping plate are arranged on the base, and a tension spring is arranged in the base, and two ends of the tension spring are fixedly connected with the left clamping plate and the right clamping plate respectively; and
a tension handle, wherein the tension handle is in transmission connection with the left clamping plate and the right clamping plate; when the tension handle is pulled inward, the left clamping plate and the right clamping plate move towards each other and compress the tension spring, and the disposable flexible ureteroscope is capable of being fixed between the left clamping plate and the right clamping plate; when the tension handle is pulled outward, the tension spring resets and separates the left clamping plate and the right clamping plate.

7. The robot of claim 1, further comprising:
a mounting frame, wherein the mounting frame is located below the bottom plate and is fixedly connected with the bottom plate;
a sheath tube feeding guide rail, wherein the sheath tube feeding guide rail is arranged on the mounting frame;
a sheath tube fixing frame, wherein the sheath tube fixing frame is arranged on the sheath tube feeding guide rail and is slidably connected with the sheath tube feeding guide rail, the ureteral guide sheath is detachably connected with the sheath tube fixing frame, and a joint between the sheath tube fixing frame and the ureteral guide sheath is close to the second side plate; and
a sheath tube feeding motor, wherein the sheath tube feeding motor is located below the mounting frame and is fixedly connected with the mounting frame, and the sheath tube feeding motor drives the sheath tube fixing frame to slide along the sheath tube feeding guide rail through a sheath tube feeding screw.

8. The robot of claim 7, further comprising:
a sheath tube rapid assembly device comprising:
a lower cover, wherein the lower cover is fixedly arranged on the sheath tube fixing frame;
an upper cover, wherein the upper cover is hinged with the lower cover, and a clamping groove adapted to the ureteral guide sheath is defined between the upper cover and the lower cover; and
a cover lock, wherein the cover lock is hinged with the lower cover, and when the upper cover rotates to an upper surface of the lower cover, the cover lock is capable of rotating to an upper surface of the upper cover.

9. The robot of claim 1, further comprising:
a support plate, wherein the bottom plate is fixedly connected to the support plate, and a bottom of the support plate extends downward to form a support; the support plate has a clamping part, the bottom plate is fixed in the clamping part, and a clamping degree of the clamping part is adjusted by a tension knob; and
a lifter, wherein the lifter is in transmission connection with the support and is capable of driving the support to move in an up-and-down direction.

10. The robot of claim 1, further comprising:
a worm gear motor, wherein the worm gear motor is fixed on the disposable flexible ureteroscope through a guide wire motor fixing plate;
a lower roller, wherein the lower roller is rotatably connected to the guide wire motor fixing plate through a lower roller cover, and the lower roller is fixedly connected with an output end of the worm gear motor;
an upper roller, wherein the upper roller is rotatably connected to the guide wire motor fixing plate through an upper roller cover, the lower roller is rotatably connected to the upper roller, a groove is defined between the lower roller and the upper roller, and a laser guide wire penetrates through the groove, and the laser guide wire is driven to feed via rotation of the lower roller and the upper roller; and
a tee fitting, wherein the tee fitting is in communication with the flexible ureteroscope tube of the disposable flexible ureteroscope, and the laser guide wire enters the flexible ureteroscope tube of the disposable flexible ureteroscope through the tee fitting.
